(19) United States
(12) Patent Application Publication
(10) Pub. No.: US 2015/0017236 A1
(43) Pub. Date: Jan. 15, 2015

(54) PHARMACEUTICAL COMPOSITIONS OF THYROID HORMONE

(71) Applicant: Jefferson J. GREGORY, Bristol, TN (US)

(72) Inventor: Jefferson J. GREGORY, Bristol, TN (US)

(21) Appl. No.: 14/326,257

(22) Filed: Jul. 8, 2014

Related U.S. Application Data
(60) Provisional application No. 61/844,403, filed on Jul. 9, 2013.

(51) Int. Cl. A61K 9/14; A61K 9/48; A61K 9/20; A61K 31/198

(52) U.S. Cl. CPC A61K 9/14; A61K 31/198; A61K 9/48; A61K 9/20
USPC 424/451; 424/490; 514/567; 424/464

(57) ABSTRACT

Provided herein are methods of stabilizing thyroid hormone, preventing a thyroid hormone from being oxidized, producing a formulation of thyroid hormone having a uniform potency, and reducing the exposure of a thyroid hormone to oxygen, light, moisture, or high temperature. Such methods comprise coating said thyroid hormone to provide a solid form comprising thyroid hormone coated with a coating material. Also provided are pharmaceutical compositions comprising a solid form of a thyroid hormone, wherein said solid form comprises thyroid hormone coated with a coating material. The invention also provides pharmaceutical compositions produced by the methods. Unit dosage forms and batches comprising such pharmaceutical compositions are also provided.

### CROSS-REFERENCE TO RELATED APPLICATIONS

This application claims priority benefit under 35 U.S.C. § 119(e) to U.S. Provisional Application Ser. No. 61/844,403, filed on Jul. 9, 2013, which is hereby incorporated by reference in its entirety.

### BACKGROUND OF THE INVENTION

Hypothyroidism is a common endocrine disorder resulting from deficiency of thyroid hormone. In the United States, one child out of every five thousand is hypothyroidic. In people aged sixty or older, the incidence of hypothyroidism increases to reach as far as 2.7% in men and 7.1% in women. Levothyroxine sodium is a widely used thyroid hormone in the treatment and/or prophylaxis of thyroid hormone disorders. A precise dosage is critical, as an underdosage can lead to a sub-optimal response, whereas overdosage can lead to toxic symptoms of hyperthyroidism, such as heart pains, palpitations or heart arrhythmias.

Formulations containing levothyroxine sodium have been known in the art since the late 1950s. However, pharmaceutical formulations containing levothyroxine sodium exhibit a relatively short shelf life, particularly in the presence of oxygen, light, humidity, or high temperature (see Won (1992) Pharmaceutical Research. 9: 131-137). At least in tablet formulation of levothyroxine sodium, this relatively short shelf life can be attributed to the decomposition of levothyroxine by approximately 1 percent per month. See Gupta et al. (1990) J. Clin. Pharm. Ther: 15:331-335. Due to the degradation of the active ingredient in currently available formulations of levothyroxine sodium, new methods of formulating solid dosage forms of this drug would be highly desirable.

All references cited herein, including patent applications and publications, are hereby incorporated by reference in their entirety.

### BRIEF SUMMARY OF THE INVENTION

The invention provided herein discloses, inter alia, a method of stabilizing thyroid hormone, wherein the method comprises coating said thyroid hormone to provide a solid form comprising thyroid hormone coated with a coating material. In some embodiments, the method comprises an air-suspension encapsulation process (e.g., the Wurster process). In some embodiments, the method comprises spraying a solid form comprising thyroid hormone with a composition comprising a pharmaceutically acceptable coating material. In some embodiments, the method comprises dissolving or dispersing the thyroid hormone in a vehicle, and spray coating the dissolved or dispersed thyroid hormone onto an inert solid dosage core. In some embodiments, the method comprises admixing the thyroid hormone in a suitable vehicle to form a mixture; spraying the mixture onto an inert core to form a thyroid hormone-coated core; and spraying a coating material onto the thyroid hormone-coated core to provide a solid form comprising thyroid hormone coated with a coating material. In some embodiments, the method comprises dissolving or dispersing the thyroid hormone in a vehicle and spraying the dissolved or dispersed thyroid hormone into a dessicant system into which a second dispersion comprising a coating mixture is being simultaneously sprayed. In some embodiments, the method comprises simultaneously spraying a first composition comprising thyroid hormone with a second composition comprising a coating material, and directing the simultaneous spray into a dessicator to provide a solid form comprising thyroid hormone coated with a coating material. In some embodiments, the method comprises admixing the thyroid hormone with a coating material, and compressing the mixture to provide a solid form comprising thyroid hormone coated with a coating material. In some of the embodiments herein, the thyroid hormone is levothyroxine or a salt thereof. In some of the embodiments herein, the thyroid hormone is levothyroxine sodium. In some of the embodiments herein, the thyroid hormone is in a hydrated form. In some embodiments herein, the hydrated form is a pentahydrate form, a dihemihydrate form or a monohydrate form. In some of the embodiments herein, the coating material is water insoluble at neutral pH. In some of the embodiments herein, the coating material is essentially gelatin-free. In some of the embodiments herein, the coating material is impermeable to one or more selected from the group consisting of: oxygen, light, and moisture. In some of the embodiments herein, the coating material is essentially oxygen-free. In some of the embodiments herein, the coating material has a minimal effect on the dissolution profile of the thyroid hormone. In some embodiments herein, the thyroid hormone is uniformly distributed in the solid form comprising thyroid hormone coated with a coating material. In some embodiments herein, the solid form comprising thyroid hormone coated with a coating material comprises a uniform matrix. In further embodiments, the uniform matrix comprises one or more of a pharmaceutically acceptable excipient selected from the group consisting of: a diluent, a filler, a binder, a disintegrant, a lubricant, a preservative, a plasticizer, and a coloring agent. In some embodiments herein, the solid form comprising thyroid hormone coated with a coating material is in a powder form. In some embodiments herein, the solid form comprising thyroid hormone coated with a coating material is in a unit dosage form. In further embodiments, the unit dosage form is a tablet. In other further embodiments, the unit dosage form is a capsule. In some embodiments herein, the solid form comprising thyroid hormone coated with a coating material is in a batch form. In some embodiments herein, the solid form comprising thyroid hormone coated with a coating material dissolves in a fluid comprising a pH from about 1 to 5. In further embodiments, the pH is from about 1 to 3. In some embodiments herein, the solid form comprising thyroid hormone coated with a coating material dissolves in the presence of an enzyme. In some embodiments herein, the solid form comprising thyroid hormone coated with a coating material is stabilized for at least about 3 months. In some embodiments herein, the solid form comprising thyroid hormone coated with a coating material is stabilized for at least about 18 months. In some embodiments herein, the solid form comprising thyroid hormone coated with a coating material has a potency loss of no more than about 10% when stored for at least 3 months. In some embodiments herein, the solid form comprising thyroid hormone coated with a coating material has a potency loss of no more than about 5% when stored for at least 3 months. In some embodiments herein, the solid form comprising thyroid hormone coated with a coating material has a particle size substantially similar to a particle size of an uncoated pharmaceutical composition comprising thyroid hormone. In some embodiments herein, the solid form comprising thyroid hormone coated with a coating material has a dissolution rate substantially similar to a dissolution rate of an uncoated pharmaceutical composition comprising thyroid hormone. In some embodiments herein, the solid form comprising thyroid hormone coated with a coating material has a bioavailability profile substantially similar to a bioavailability profile of an uncoated pharmaceutical composition comprising thyroid hormone. In some embodiments herein, the solid form comprising thyroid hormone coated with a coating material has a therapeutic effect substantially similar to a therapeutic effect of an uncoated pharmaceutical composition comprising thyroid hormone. In some of the embodiments herein, the solid form comprising thyroid hormone coated with a coating material has an increased shelf-life.

In another aspect, the invention provided herein discloses a method of preventing a thyroid hormone from being oxidized or degraded, wherein the method comprises coating said thyroid hormone to provide a solid form comprising thyroid hormone coated with a coating material. In some embodiments, the method comprises an air-suspension encapsulation process (e.g., the Wurster process). In some embodiments, the method comprises spraying a solid form comprising thyroid hormone with a composition comprising a pharmaceutically acceptable coating material. In some embodiments, the method comprises dissolving or dispersing the thyroid hormone in a vehicle, and spray coating the dissolved or dispersed thyroid hormone onto an inert solid dosage core. In some embodiments, the method comprises admixing the thyroid hormone in a suitable vehicle to form a mixture; spraying the mixture onto an inert core to form a thyroid hormone-coated core; and spraying a coating material onto the thyroid hormone-coated core to provide a solid form comprising thyroid hormone coated with a coating material. In some embodiments, the method comprises dissolving or dispersing the thyroid hormone in a vehicle and spraying the dissolved or dispersed thyroid hormone into a dessicant system into which a second dispersion comprising a coating mixture is being simultaneously sprayed. In some embodiments, the method comprises simultaneously spraying a first composition comprising thyroid hormone with a second composition comprising a coating material, and directing the simultaneous spray into a dessicator to provide a solid form comprising thyroid hormone coated with a coating material. In some embodiments, the method comprises admixing the thyroid hormone with a coating material, and compressing the mixture to provide a solid form comprising thyroid hormone coated with a coating material. In some of the embodiments herein, the thyroid hormone is levothyroxine or a salt thereof. In some of the embodiments herein, the thyroid hormone is levothyroxine sodium. In some of the embodiments herein, the thyroid hormone is in a hydrated form. In some embodiments herein, the hydrated form is a pentahydrate form, a dihemihydrate form or a monohydrate form. In some of the embodiments herein, the coating material is water insoluble at neutral pH. In some of the embodiments herein, the coating material is essentially gelatin-free. In some of the embodiments herein, the coating material is impermeable to one or more selected from the group consisting of: oxygen, light, and moisture. In some of the embodiments herein, the coating material is essentially oxygen-free. In some of the embodiments herein, the coating material has a minimal effect on the dissolution profile of the thyroid hormone. In some embodiments herein, the thyroid hormone is uniformly distributed in the solid form comprising thyroid hormone coated with a coating material. In some embodiments herein, the solid form comprising thyroid hormone coated with a coating material comprises a uniform matrix. In further embodiments, the uniform matrix comprises one or more of a pharmaceutically acceptable excipient selected from the group consisting of: a diluent, a filler, a binder, a disintegrant, a lubricant, a preservative, a plasticizer, and a coloring agent. In some embodiments herein, the solid form comprising thyroid hormone coated with a coating material is in a powder form. In some embodiments herein, the solid form comprising thyroid hormone coated with a coating material is in a unit dosage form. In further embodiments, the unit dosage form is a tablet. In other further embodiments, the unit dosage form is a capsule. In some embodiments herein, the solid form comprising thyroid hormone coated with a coating material is in a batch form. In some embodiments herein, the solid form comprising thyroid hormone coated with a coating material dissolves in a fluid comprising a pH from about 1 to 5. In further embodiments, the pH is from about 1 to 3. In some embodiments herein, the solid form comprising thyroid hormone coated with a coating material dissolves in the presence of an enzyme. In some embodiments herein, the solid form comprising thyroid hormone coated with a coating material is stabilized for at least about 3 months. In some embodiments herein, the solid form comprising thyroid hormone coated with a coating material is stabilized for at least about 18 months. In some embodiments herein, the solid form comprising thyroid hormone coated with a coating material has a potency loss of no more than about 10% when stored for at least 3 months. In some embodiments herein, the solid form comprising thyroid hormone coated with a coating material has a potency loss of no more than about 5% when stored for at least 3 months. In some embodiments herein, the solid form comprising thyroid hormone coated with a coating material has a particle size substantially similar to a particle size of an uncoated pharmaceutical composition comprising thyroid hormone. In some embodiments herein, the solid form comprising thyroid hormone coated with a coating material has a dissolution rate substantially similar to a dissolution rate of an uncoated pharmaceutical composition comprising thyroid hormone. In some embodiments herein, the solid form comprising thyroid hormone coated with a coating material has a bioavailability profile substantially similar to a bioavailability profile of an uncoated pharmaceutical composition comprising thyroid hormone. In some embodiments herein, the solid form comprising thyroid hormone coated with a coating material has a therapeutic effect substantially similar to a therapeutic effect of an uncoated pharmaceutical composition comprising thyroid hormone. In some of the embodiments herein, the solid form comprising thyroid hormone coated with a coating material has an increased shelf-life.

In another aspect, the invention provided herein discloses a method of producing a formulation of thyroid hormone having a uniform potency, comprising coating said thyroid hormone to provide a solid form comprising thyroid hormone coated with a coating material. In some embodiments, the method comprises an air-suspension encapsulation process (e.g., the Wurster process). In some embodiments, the method comprises spraying a solid form comprising thyroid hormone with a composition comprising a pharmaceutically acceptable coating material. In some embodiments, the method comprises dissolving or dispersing the thyroid hormone in a vehicle, and spray coating the dissolved or dispersed thyroid hormone onto an inert solid dosage core. In some embodiments, the method comprises admixing the thyroid hormone in a suitable vehicle to form a mixture; spraying the mixture onto an inert core to form a thyroid hormone-coated core; and spraying a coating material onto the thyroid hormone-coated core to provide a solid form comprising thyroid hormone coated with a coating material. In some embodiments, the method comprises dissolving or dispersing the thyroid hormone in a vehicle and spraying the dissolved or dispersed thyroid hormone into a dessicant system into which a second dispersion comprising a coating mixture is being simultaneously sprayed. In some embodiments, the method comprises simultaneously spraying a first composition comprising thyroid hormone with a second composition comprising a coating material, and directing the simultaneous spray into a dessicator to provide a solid form comprising thyroid hormone coated with a coating material. In some embodiments, the method comprises admixing the thyroid hormone with a coating material, and compressing the mixture to provide a solid form comprising thyroid hormone coated with a coating material. In some of the embodiments herein, the thyroid hormone is levothyroxine or a salt thereof. In some of the embodiments herein, the thyroid hormone is levothyroxine sodium. In some of the embodiments herein, the thyroid hormone is in a hydrated form. In some embodiments herein, the hydrated form is a pentahydrate form, a dihemihydrate form or a monohydrate form. In some of the embodiments herein, the coating material is water insoluble at neutral pH. In some of the embodiments herein, the coating material is essentially gelatin-free. In some of the embodiments herein, the coating material is impermeable to one or more selected from the group consisting of: oxygen, light, and moisture. In some of the embodiments herein, the coating material is essentially oxygen-free. In some of the embodiments herein, the coating material has a minimal effect on the dissolution profile of the thyroid hormone. In some embodiments herein, the thyroid hormone is uniformly distributed in the solid form comprising thyroid hormone coated with a coating material. In some embodiments herein, the solid form comprising thyroid hormone coated with a coating material comprises a uniform matrix. In further embodiments, the uniform matrix comprises one or more of a pharmaceutically acceptable excipient selected from the group consisting of: a diluent, a filler, a binder, a disintegrant, a lubricant, a preservative, a plasticizer, and a coloring agent. In some embodiments herein, the solid form comprising thyroid hormone coated with a coating material is in a powder form. In some embodiments herein, the solid form comprising thyroid hormone coated with a coating material is in a unit dosage form. In further embodiments, the unit dosage form is a tablet. In other further embodiments, the unit dosage form is a capsule. In some embodiments herein, the solid form comprising thyroid hormone coated with a coating material is in a batch form. In some embodiments herein, the solid form comprising thyroid hormone coated with a coating material dissolves in a fluid comprising a pH from about 1 to 5. In further embodiments, the pH is from about 1 to 3. In some embodiments herein, the solid form comprising thyroid hormone coated with a coating material dissolves in the presence of an enzyme. In some embodiments herein, the solid form comprising thyroid hormone coated with a coating material is stabilized for at least about 3 months. In some embodiments herein, the solid form comprising thyroid hormone coated with a coating material is stabilized for at least about 18 months. In some embodiments herein, the solid form comprising thyroid hormone coated with a coating material has a potency loss of no more than about 10% when stored for at least 3 months. In some embodiments herein, the solid form comprising thyroid hormone coated with a coating material has a potency loss of no more than about 5% when stored for at least 3 months. In some embodiments herein, the solid form comprising thyroid hormone coated with a coating material has a particle size substantially similar to a particle size of an uncoated pharmaceutical composition comprising thyroid hormone. In some embodiments herein, the solid form comprising thyroid hormone coated with a coating material has a dissolution rate substantially similar to a dissolution rate of an uncoated pharmaceutical composition comprising thyroid hormone. In some embodiments herein, the solid form comprising thyroid hormone coated with a coating material has a bioavailability profile substantially similar to a bioavailability profile of an uncoated pharmaceutical composition comprising thyroid hormone. In some embodiments herein, the solid form comprising thyroid hormone coated with a coating material has a therapeutic effect substantially similar to a therapeutic effect of an uncoated pharmaceutical composition comprising thyroid hormone. In some of the embodiments herein, the solid form comprising thyroid hormone coated with a coating material has an increased shelf-life.

In another aspect, the invention provided herein discloses a method of reducing the exposure of a thyroid hormone to oxygen, light, moisture, or high temperature, comprising coating said thyroid hormone to provide a solid form comprising thyroid hormone coated with a coating material. In some embodiments, the method comprises an air-suspension encapsulation process (e.g., the Wurster process). In some embodiments, the method comprises spraying a solid form comprising thyroid hormone with a composition comprising a pharmaceutically acceptable coating material. In some embodiments, the method comprises dissolving or dispersing the thyroid hormone in a vehicle, and spray coating the dissolved or dispersed thyroid hormone onto an inert solid dosage core. In some embodiments, the method comprises admixing the thyroid hormone in a suitable vehicle to form a mixture; spraying the mixture onto an inert core to form a thyroid hormone-coated core; and spraying a coating material onto the thyroid hormone-coated core to provide a solid form comprising thyroid hormone coated with a coating material. In some embodiments, the method comprises dissolving or dispersing the thyroid hormone in a vehicle and spraying the dissolved or dispersed thyroid hormone into a dessicant system into which a second dispersion comprising a coating mixture is being simultaneously sprayed. In some embodiments, the method comprises simultaneously spraying a first composition comprising thyroid hormone with a second composition comprising a coating material, and directing the simultaneous spray into a dessicator to provide a solid form comprising thyroid hormone coated with a coating material. In some embodiments, the method comprises admixing the thyroid hormone with a coating material, and compressing the mixture to provide a solid form comprising thyroid hormone coated with a coating material. In some of the embodiments herein, the thyroid hormone is levothyroxine or a salt thereof. In some of the embodiments herein, the thyroid hormone is levothyroxine sodium. In some of the embodiments herein, the thyroid hormone is in a hydrated form. In some embodiments herein, the hydrated form is a pentahydrate form, a dihemihydrate form or a monohydrate form. In some of the embodiments herein, the coating material is water insoluble at neutral pH. In some of the embodiments herein, the coating material is essentially gelatin-free. In some of the embodiments herein, the coating material is impermeable to one or more selected from the group consisting of: oxygen, light, and moisture. In some of the embodiments herein, the coating material is essentially oxygen-free. In some of the embodiments herein, the coating material has a minimal effect on the dissolution profile of the thyroid hormone. In some embodiments herein, the thyroid hormone is uniformly distributed in the solid form comprising thyroid hormone coated with a coating material. In some embodiments herein, the solid form comprising thyroid hormone coated with a coating material comprises a uniform matrix. In further embodiments, the uniform matrix comprises one or more of a pharmaceutically acceptable excipient selected from the group consisting of: a diluent, a filler, a binder, a disintegrant, a lubricant, a preservative, a plasticizer, and a coloring agent. In some embodiments herein, the solid form comprising thyroid hormone coated with a coating material is in a powder form. In some embodiments herein, the solid form comprising thyroid hormone coated with a coating material is in a unit dosage form. In further embodiments, the unit dosage form is a tablet. In other further embodiments, the unit dosage form is a capsule. In some embodiments herein, the solid form comprising thyroid hormone coated with a coating material is in a batch form. In some embodiments herein, the solid form comprising thyroid hormone coated with a coating material dissolves in a fluid comprising a pH from about 1 to 5. In further embodiments, the pH is from about 1 to 3. In some embodiments herein, the solid form comprising thyroid hormone coated with a coating material dissolves in the presence of an enzyme. In some embodiments herein, the solid form comprising thyroid hormone coated with a coating material is stabilized for at least about 3 months. In some embodiments herein, the solid form comprising thyroid hormone coated with a coating material is stabilized for at least about 18 months. In some embodiments herein, the solid form comprising thyroid hormone coated with a coating material has a potency loss of no more than about 10% when stored for at least 3 months. In some embodiments herein, the solid form comprising thyroid hormone coated with a coating material has a potency loss of no more than about 5% when stored for at least 3 months. In some embodiments herein, the solid form comprising thyroid hormone coated with a coating material has a particle size substantially similar to a particle size of an uncoated pharmaceutical composition comprising thyroid hormone. In some embodiments herein, the solid form comprising thyroid hormone coated with a coating material has a dissolution rate substantially similar to a dissolution rate of an uncoated pharmaceutical composition comprising thyroid hormone. In some embodiments herein, the solid form comprising thyroid hormone coated with a coating material has a bioavailability profile substantially similar to a bioavailability profile of an uncoated pharmaceutical composition comprising thyroid hormone. In some embodiments herein, the solid form comprising thyroid hormone coated with a coating material has a therapeutic effect substantially similar to a therapeutic effect of an uncoated pharmaceutical composition comprising thyroid hormone. In some of the embodiments herein, the solid form comprising thyroid hormone coated with a coating material has an increased shelf-life.

In yet another aspect, the invention provided herein discloses a pharmaceutical composition comprising thyroid hormone produced by any of the methods described herein. In some embodiments, the pharmaceutical composition is produced by a method comprising an air-suspension encapsulation process (e.g., the Wurster process). In some embodiments, the pharmaceutical composition is produced by a method comprising spraying a solid form comprising thyroid hormone with a composition comprising a pharmaceutically acceptable coating material. In some embodiments, the pharmaceutical composition is produced by a method comprising dissolving or dispersing the thyroid hormone in a vehicle, and spray coating the dissolved or dispersed thyroid hormone onto an inert solid dosage core. In some embodiments, the pharmaceutical composition is produced by a method comprising admixing the thyroid hormone in a suitable vehicle to form a mixture; spraying the mixture onto an inert core to form a thyroid hormone-coated core; and spraying a coating material onto the thyroid hormone-coated core to provide a solid form comprising thyroid hormone coated with a coating material. In some embodiments, the pharmaceutical composition is produced by a method comprising dissolving or dispersing the thyroid hormone in a vehicle and spraying the dissolved or dispersed thyroid hormone into a dessicant system into which a second dispersion comprising a coating mixture is being simultaneously sprayed. In some embodiments, the pharmaceutical composition is produced by a method comprising simultaneously spraying a first composition comprising thyroid hormone with a second composition comprising a coating material, and directing the simultaneous spray into a dessicator to provide a solid form comprising thyroid hormone coated with a coating material. In some embodiments, the pharmaceutical composition is produced by a method comprising admixing the thyroid hormone with a coating material, and compressing the mixture to provide. In some of the embodiments herein, the thyroid hormone is levothyroxine or a salt thereof. In some of the embodiments herein, the thyroid hormone is levothyroxine sodium. In some of the embodiments herein, the thyroid hormone is in a hydrated form. In some embodiments herein, the hydrated form is a pentahydrate form, a dihemihydrate form or a monohydrate form. In some of the embodiments herein, the coating material is water insoluble at neutral pH. In some of the embodiments herein, the coating material is essentially gelatin-free. In some of the embodiments herein, the coating material is impermeable to one or more selected from the group consisting of: oxygen, light, and moisture. In some of the embodiments herein, the coating material is essentially oxygen-free. In some of the embodiments herein, the coating material has a minimal effect on the dissolution profile of the thyroid hormone. In some embodiments herein, the thyroid hormone is uniformly distributed in the solid form comprising thyroid hormone coated with a coating material. In some embodiments herein, the pharmaceutical composition comprising thyroid hormone coated with a coating material comprises a uniform matrix. In further embodiments, the uniform matrix comprises one or more of a pharmaceutically acceptable excipient selected from the group consisting of: a diluent, a filler, a binder, a disintegrant, a lubricant, a preservative, a plasticizer, and a coloring agent. In some embodiments herein, the pharmaceutical composition comprising thyroid hormone coated with a coating material is in a powder form. In some embodiments herein, the pharmaceutical composition comprising thyroid hormone coated with a coating material is in a unit dosage form. In further embodiments, the unit dosage form is a tablet. In other further embodiments, the unit dosage form is a capsule. In some embodiments herein, the pharmaceutical composition comprising thyroid hormone coated with a coating material is in a batch form. In some embodiments herein, the pharmaceutical composition comprising thyroid hormone coated with a coating material dissolves in a fluid comprising a pH from about 1 to 5. In further embodiments, the pH is from about 1 to 3. In some embodiments herein, the solid form comprising thyroid hormone coated with a coating material dissolves in the presence of an enzyme. In some embodiments herein, the pharmaceutical composition comprising thyroid hormone coated with a coating material is stabilized for at least about 3 months. In some embodiments herein, the pharmaceutical composition comprising thyroid hormone coated with a coating material is stabilized for at least about 18 months. In some embodiments herein, the pharmaceutical composition comprising thyroid hormone coated with a coating material has a potency loss of no more than about 10% when stored for at least 3 months. In some embodiments herein, the pharmaceutical composition comprising thyroid hormone coated with a coating material has a potency loss of no more than about 5% when stored for at least 3 months. In some embodiments herein, the pharmaceutical composition comprising thyroid hormone coated with a coating material has a particle size substantially similar to a particle size of an uncoated pharmaceutical composition comprising thyroid hormone. In some embodiments herein, the pharmaceutical composition comprising thyroid hormone coated with a coating material has a dissolution rate substantially similar to a dissolution rate of an uncoated pharmaceutical composition comprising thyroid hormone. In some embodiments herein, the pharmaceutical composition comprising thyroid hormone coated with a coating material has a bioavailability profile substantially similar to a bioavailability profile of an uncoated pharmaceutical composition comprising thyroid hormone. In some embodiments herein, the pharmaceutical composition comprising thyroid hormone coated with a coating material has a therapeutic effect substantially similar to a therapeutic effect of an uncoated pharmaceutical composition comprising thyroid hormone. In some of the embodiments herein, the pharmaceutical composition comprising thyroid hormone coated with a coating material has an increased shelf-life.

In another aspect, the invention provided herein discloses a pharmaceutical composition comprising a solid form of a thyroid hormone, wherein said solid form comprises thyroid hormone coated with a coating material. In some embodiments, the pharmaceutical composition comprises an effective amount of a thyroid hormone, and a pharmaceutically acceptable coating material, wherein the pharmaceutical acceptable coating encloses the thyroid hormone to provide a solid form of the thyroid hormone substantially free of oxygen scavenger. In some of the embodiments herein, the thyroid hormone is levothyroxine or a salt thereof. In some of the embodiments herein, the thyroid hormone is levothyroxine sodium. In some of the embodiments herein, the thyroid hormone is in a hydrated form. In some embodiments herein, the hydrated form is a pentahydrate form, a dihemihydrate form or a monohydrate form. In some of the embodiments herein, the coating material is water insoluble at neutral pH. In some of the embodiments herein, the coating material is essentially gelatin-free. In some of the embodiments herein, the coating material is impermeable to one or more selected from the group consisting of: oxygen, light, and moisture. In some of the embodiments herein, the coating material is essentially oxygen-free. In some of the embodiments herein, the coating material has a minimal effect on the dissolution profile of the thyroid hormone. In some embodiments herein, the thyroid hormone is uniformly distributed in the solid form comprising thyroid hormone coated with a coating material. In some embodiments herein, the pharmaceutical composition comprising thyroid hormone coated with a coating material comprises a uniform matrix. In further embodiments, the uniform matrix comprises one or more of a pharmaceutically acceptable excipient selected from the group consisting of: a diluent, a filler, a binder, a disintegrant, a lubricant, a preservative, a plasticizer, and a coloring agent. In some embodiments herein, the pharmaceutical composition comprising thyroid hormone coated with a coating material is in a powder form. In some embodiments herein, the pharmaceutical composition comprising thyroid hormone coated with a coating material is in a unit dosage form. In further embodiments, the unit dosage form is a tablet. In other further embodiments, the unit dosage form is a capsule. In some embodiments herein, the pharmaceutical composition comprising thyroid hormone coated with a coating material is in a batch form. In some embodiments herein, the pharmaceutical composition comprising thyroid hormone coated with a coating material dissolves in a fluid comprising a pH from about 1 to 5. In further embodiments, the pH is from about 1 to 3. In some embodiments herein, the solid form comprising thyroid hormone coated with a coating material dissolves in the presence of an enzyme. In some embodiments herein, the pharmaceutical composition comprising thyroid hormone coated with a coating material is stabilized for at least about 3 months. In some embodiments herein, the pharmaceutical composition comprising thyroid hormone coated with a coating material is stabilized for at least about 18 months. In some embodiments herein, the pharmaceutical composition comprising thyroid hormone coated with a coating material has a potency loss of no more than about 10% when stored for at least 3 months. In some embodiments herein, the pharmaceutical composition comprising thyroid hormone coated with a coating material has a potency loss of no more than about 5% when stored for at least 3 months. In some embodiments herein, the pharmaceutical composition comprising thyroid hormone coated with a coating material has a particle size substantially similar to a particle size of an uncoated pharmaceutical composition comprising thyroid hormone. In some embodiments herein, the pharmaceutical composition comprising thyroid hormone coated with a coating material has a dissolution rate substantially similar to a dissolution rate of an uncoated pharmaceutical composition comprising thyroid hormone. In some embodiments herein, the pharmaceutical composition comprising thyroid hormone coated with a coating material has a bioavailability profile substantially similar to a bioavailability profile of an uncoated pharmaceutical composition comprising thyroid hormone. In some embodiments herein, the pharmaceutical composition comprising thyroid hormone coated with a coating material has a therapeutic effect substantially similar to a therapeutic effect of an uncoated pharmaceutical composition comprising thyroid hormone. In some of the embodiments herein, the pharmaceutical composition comprising thyroid hormone coated with a coating material has an increased shelf-life.

In yet another aspect, the invention provided herein discloses a unit dosage form comprising any one of the pharmaceutical compositions described herein. In some embodiments, the unit dosage form is a tablet. In some embodiments, the unit dosage form is a capsule. In some embodiments, the unit dosage form is in a sealed container. In some embodiments, the unit dosage form is in an unsealed container.

In another aspect, the invention herein also provides a batch comprising any one of the pharmaceutical compositions described herein.

In still another aspect, the invention provides a kit comprising any unit dosage form described herein.

It is to be understood that one, some, or all of the properties of the various embodiments described herein may be combined to form other embodiments of the present invention. These and other aspects of the invention will become apparent to one of skill in the art.

### DETAILED DESCRIPTION OF THE INVENTION

The present invention in some embodiments provides a method of stabilizing a thyroid hormone (such as levothyroxine or salt thereof) by coating the thyroid hormone with a coating material. Such coating material reduces (and in some embodiments prevents) the exposure of the thyroid hormone to oxygen, light, moisture, and/or heat, thus reducing the tendency of the thyroid hormone to be oxidized and/or degraded. The methods described herein thus allow the production of solid dosage forms of thyroid hormone that are more stable and more uniform in potency.

Thus, the present invention in one aspect provides a method of stabilizing a thyroid hormone comprising coating said thyroid hormone to provide a solid form comprising thyroid hormone coated with a coating material. Also provided are formulations made by such method.

In another aspect, there is provided a formulation (including unit dosage forms and batches) comprising a solid form of thyroid hormone, wherein said solid form comprises thyroid hormone coated with a coating material.

Also provided are kits and articles of manufacture (such as a vial, for example an unsealed vial) comprising the formulations disclosed therein, as well of uses of the formulations disclosed therein.

It is understood that the term “thyroid hormone” encompasses a thyroid hormone and salts thereof (e.g., levothyroxine and salts thereof such as levothyroxine sodium).

### Methods of Stabilizing Thyroid Hormone

The present invention provides methods of stabilizing thyroid hormone by coating said thyroid hormone to provide a solid form comprising thyroid hormone coated with a coating material. The coating in some embodiments reduces (and in some embodiments prevents) exposure of the thyroid hormone to oxygen, moisture, light, and/or high temperature (such as high ambient temperature). The methods described herein may reduce the risk of the thyroid hormone being oxidized and/or degraded, thus allowing the maintenance of potency of the thyroid hormone and uniformity among different dosage units.

Thus, for example, in some embodiments, there is provided a method of preventing a thyroid hormone from being oxidized, comprising coating said thyroid hormone to provide a solid form comprising thyroid hormone coated with a coating material. In some embodiments, the thyroid hormone in a solid form coated with a coating material is least about 1% less, 2% less, at least about 3% less, at least about 5% less, at least about 10% less, at least about 15% less, at least about 20% less, at least about 25% less, at least about 30% less, at least about 35% less, at least about 40% less, at least about 45% less, at least about 50% less, at least about 55% less, at least about 60% less, at least about 65% less, at least about 70% less, at least about 85% less, at least about 90% less, at least about 95% less or more than 95% less oxidized than the thyroid hormone in a solid form that is not coated with a coating material, including any range in between these values.

In some embodiments, there is provided a method of preventing a thyroid hormone from being degraded, comprising coating said thyroid hormone to provide a solid form comprising thyroid hormone coated with a coating material. In some embodiments, the thyroid hormone in a solid form coated with a coating material is least about 1% less, at least about 2% less, at least about 3% less, at least about 5% less, at least about 10% less, at least about 15% less, at least about 20% less, at least about 25% less, at least about 30% less, at least about 35% less, at least about 40% less, at least about 45% less, at least about 50% less, at least about 55% less, at least about 60% less, at least about 65% less, at least about 70% less, at least about 85% less, at least about 90% less, at least about 95% less or more than 95% less degraded than the thyroid hormone in a solid form that is not coated with a coating material, including any range in between these values.

In some embodiments, there is provided a method of producing a pharmaceutical composition comprising thyroid hormone having a uniform potency (for example a potency with less than about any of 6%, 5%, 4%, 3%, 2%, or 1% deviation), comprising coating said thyroid hormone to provide a solid form comprising thyroid hormone coated with a coating material. In some embodiments, the thyroid hormone in a solid form coated with a coating material has a consistent potency over an extended shelf life compared to thyroid hormone in a solid form that is not coated with a coating material. In some embodiments, the potency of the solid form comprising thyroid hormone coated with a coating material is than about 6%, less than about 5.5%, less than about 5%, less than about 4.5%, less than about 4%, less than about 3.5%, less than about 3%, less than about 2.5%, less than about 2%, less than about 1.75%, less than about 1.5%, less than about 1%, less than about 0.75%, less than about 0.5%, less than 0.4% deviation, less than about 0.3%, less than about 0.2%, or less than about 0.1% deviation as compared to thyroid hormone in a solid form that is not coated with a coating material.

In some embodiments, there is provided a method of increasing shelf life of a thyroid hormone, comprising coating said thyroid hormone to provide a solid form comprising thyroid hormone coated with a coating material. In some embodiments, the shelf life of the thyroid hormone in a solid form coated with a coating material is longer than thyroid hormone in a solid form that is not coated with a coating material. In some embodiments, the shelf life of the thyroid hormone in a solid form coated with a coating material is longer under accelerated storage conditions (i.e., at increased temperatures, in the presence of light, under conditions of increased relative humidity, etc.) than thyroid hormone in a solid form that is not coated with a coating material. In some embodiments, the shelf life of the thyroid hormone in a solid form coated with a coating material is at least 15 months. For example, in some embodiments, the shelf life of the thyroid hormone in a solid form coated with a coating material is at least 15 months, at least 18 months, at least 21 months, at least 24 months, at least 27 months, at least 30 months, at least 33 months, at least 36 months, or greater than 36 months, but no more than 60 months, including any range in between these values. In some embodiments, In some embodiments, the shelf life of the thyroid hormone in a solid form coated with a coating material is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 90%, at least 95%, at least 100%, at least 125%, at least 150%, at least 175%, at least 200%, or more than 200% longer than the shelf life of thyroid hormone in a solid form that is not coated with a coating material.

In some embodiments, there is provided a method of reducing (and in some embodiments preventing) the exposure of a thyroid hormone to oxygen, comprising coating said thyroid hormone to provide a solid form comprising thyroid hormone coated with a coating material. In some embodiments, there is provided a method of reducing (and in some embodiments preventing) the exposure of a thyroid hormone to moisture, comprising coating said thyroid hormone to provide a solid form comprising thyroid hormone coated with a coating material. In some embodiments, there is provided a method of reducing (and in some embodiments preventing) the exposure of a thyroid hormone to light, comprising coating said thyroid hormone to provide a solid form comprising thyroid hormone coated with a coating material. In some embodiments, there is provided a method of reducing (and in some embodiments preventing) the exposure of a thyroid hormone to a high temperature (such as a high ambient temperature), comprising coating said thyroid hormone to provide a solid form comprising thyroid hormone coated with a coating material.

The thyroid hormone can be coated with the coating material by a variety of methods. In some embodiments, the method comprises microencapsulation. In one exemplary process, the thyroid hormone (e.g., levothyroxine or salt thereof) can be coated via air-suspension encapsulation, such as, for example, the Wurster fluid bed process (see, e.g., Rajesh et al. (2012) “Wurster coating—Process and Product variables.” Int J. Pharm. Inn. 2(2): 61-66). This process takes place in a Wurster machine, i.e., a fluid bed that is divided into two zones by a partition that organizes the flow of particles. The Wurster machine is characterized by a cylindrical product container with perforated disc at the bottom. Inside the product container, a second cylinder (coating partition) is raised slightly above the perforated disk. In the center of the plate, a spray nozzle for dispensing the coating material is fitted below the coating partition. The perforated disc is designed with the large hole under the coating partition and one ring at the perimeter, and smaller holes at the remainder of the plate. This configuration permits particles, such as particles of thyroid hormone (for example levothyroxine sodium), to be pneumatically accelerated towards the upward through the coating partition upward past the spray nozzle. In some embodiments, the particles are between about 5 μm and 50 μm in diameter, such as about 7 μm, about 10 μm, about 20 μm, about 30 μm, about 40 μm, or but no more than about 50 μm in diameter, including any range in between these values. The nozzle sprays atomized droplets of coating material concurrently with particle flow. Passing particles move upward into an expansion chamber as droplets deposit on their surfaces. The coating dries as the particles are suspended, to prevent agglomeration and accretion. The particles the fall back into the outer section of the perforated plate, where they are drawn back into the high velocity air stream, repeating the cycle. During each pass through the coating partition the substrate particles receives an increment of coating material. The particles circulate rapidly as cycles are repeated. In certain embodiments, the cycles are repeated up to about several hundred times to achieve the coating thickness desired. The quality of the coating applied can be controlled by monitoring key process and product variables known in the art. In some embodiments, a complete sealing of the surface of a thyroid hormone particle can be achieved.

In some embodiments, the method comprises spraying a solid dosage form comprising the thyroid hormone with a composition comprising a pharmaceutically acceptable coating material.

For example, solid dosage forms of thyroid hormone (such as levothyroxine, e.g., levothyroxine tablets), produced according to any one of a variety of methods known in the art, can be coated to increase their stability. Commercially used technologies for coating solid dosage forms entail dissolving a mixture of polymers (such as acrylic polymers or copolymers, plastic polymers, latex polymers, or cellulose derivatives), pigments and/or excipients in an appropriate organic solvent (such as methylene chloride, glycerol, propanol, butanol, methanol, ethyl alcohol, isopropyl alcohol, methylene dichloride, acetone, chloroform, or blends thereof) to form a solution and spraying the solution onto the solid dosage forms thyroid hormone. The solid dosage forms are then dried continuously until a dry coating film is formed. The liquid coating processes and equipment have been well established and widely adopted by the pharmaceutical industry. See, e.g., Srivastava et al. (2010) Int J Pharm Sci 2: 236-246. Typical liquid coating is carried out in a rotary pan coater for larger size solid dosages such as tablets, or in a fluidized bed coater for smaller size dosage forms such as pellets or pills. Further details regarding coating solid dosage forms are described in, e.g., Cole G. Pharmaceutical Coating Technology, Taylor and Francis Ltd, 1998;1-5; Porter C. Coating of Pharmaceutical Solid-dosage forms, Pharm. Tech., 1980,4(3), 66; Hinkes T, Solvent film coating: aqueous vs. Organic, Wisconsin Alumni Research Foundation Madison, Wisconsin; Porter S. Coating of Pharmaceutical Dosage forms, Remington's book of science, 1(46); 894-902; and others.

In certain embodiments, the thyroid hormone that has been dissolved or dispersed is spray coated onto an inert solid dosage core. In one exemplary method, a thyroid hormone (such as levothyroxine, e.g., levothyroxine sodium) can be dissolved or dispersed in a suitable vehicle (such as an organic solvent, including, but not limited to methylene chloride, glycerol, propanol, butanol, methanol, ethyl alcohol, isopropyl alcohol, methylene dichloride, acetone, chloroform, or blends thereof) and spray coated onto an inert core, e.g., inert tablet cores, inert beads, or inert spheres. Non-limiting examples of various substances that can be used for inert cores include insoluble inert materials such as plastic particles/beads or silicon dioxide, calcium phosphate dihydrate, dicalcium phosphate, calcium sulfate dihydrate, microcrystalline cellulose, cellulose derivatives, calcium carbonate, dibasic calcium phosphate anhydrous, dibasic calcium phosphate monohydrate, tribasic calcium phosphate, magnesium carbonate, and magnesium oxide, soluble cores such as sugar spheres having sugars like dextrose, lactose, anhydrous lactose, spray-dried lactose, lactose monohydrate, mannitol, starches, sorbitol, and sucrose, insoluble inert plastic materials such as spherical or nearly spherical core beads of polyvinylchloride or polystyrene, and any other pharmaceutically acceptable insoluble synthetic materials, and the like and mixtures thereof. The thyroid hormone-coated inert cores may be further coated, e.g., spray coated, with a coating material, producing a particle in which a layer of thyroid hormone (e.g., levothyroxine or salt thereof) is sandwiched between the inert core and the external coating material. The coating material preferably stabilizes the thyroid hormone, prevents the thyroid hormone from being oxidized, maintains the thyroid hormone's potency and/or reduces the exposure of the thyroid hormone to light, moisture, oxygen, or high temperature.

In some embodiments, the method comprises simultaneously spraying a first composition comprising thyroid hormone with a second composition comprising a pharmaceutically acceptable coating material to form a stable coated composition of the thyroid hormone.

In some embodiments, the thyroid hormone can be dissolved or dispersed in an appropriate vehicle, such as an organic solvent (e.g., methylene chloride, glycerol, propanol, butanol, methanol, ethyl alcohol, isopropyl alcohol, methylene dichloride, acetone, chloroform, or blends thereof), to form a solution, emulsion, slurry, or paste that is capable of being atomized. The thyroid hormone mixture is then spray dried into a dessicant system into which a second dispersion comprising a coating mixture, e.g., comprising plastic or polymer, is being simultaneously sprayed. As the vehicle in which the thyroid hormone is dissolved or dispersed evaporates, the coating material envelops the suspended thyroid hormone particles. Alternatively, the thyroid hormone can be dissolved or dispersed in a vehicle that comprises the coating material and spray dried according to methods known in the art. See, e.g., Parikh D, Spray drying as a granulation Technique; In: Handbook of Pharmaceutical Granulation Technology, Drugs and the Pharmaceutical Sciences. New York, Marcel Dekker. 1997; 75-96; Maria-Inês Rê. Formulating Drug Delivery Systems by Spray Drying, Drying Technology 2006, 24, 433-446; Christensen K, Pedersen G, Kristensen H. Preparation of redispersible dry emulsions by spray drying, International Journal of Pharmaceutics 2001, 212, 187-194; and others. The coated thyroid hormone particles can then be mixed with one or more appropriate diluents, binders, lubricants, colorants, disintegrants, and/or surfactants and processed into solid pharmaceutical dosage forms according to methods known in the art, such as compression, dry or wet granulation, extrusion, etc.

The compositions produced by the methods described herein, including the coating materials and the thyroid hormones used for the methods described herein, are discussed further below.

Compositions of the present application

Also provided herein are pharmaceutical compositions comprising a thyroid hormone coated with a coating material, including pharmaceutical compositions produced by any one of the methods described herein.

For example, in some embodiments, there is provided a pharmaceutical composition comprising a solid form of thyroid hormone, wherein said solid form comprises thyroid hormone coated with a coating material. In some embodiments, the thyroid hormone is levothyroxine or a salt thereof. In some embodiments, the thyroid hormone is levothyroxine. In some embodiments, there is provided a pharmaceutical composition comprising a solid form of thyroid hormone, wherein said solid form comprises thyroid hormone coated with a coating material, wherein the solid form in the pharmaceutical composition has a uniform potency (for example a potency with less than about any of 6%, 5%, 4%, 3%, 2%, or 1% deviation).

In some embodiments, there is provided a pharmaceutical composition comprising a solid form of thyroid hormone, wherein said solid form comprises thyroid hormone coated with a coating material, wherein the thyroid hormone is evenly distributed within the coating material.

In some embodiments, the solid form is in the form of a powder. In some embodiments, the solid form is in the form of a tablet. In some embodiments, the solid form is in the form of particles (such as microparticles or nanoparticles). In some embodiments, the solid form is in the form of a capsule. In some embodiments, the solid form comprises the thyroid hormone distributed (for example evenly distributed) within a matrix, which in turn is coated with the coating material. In some embodiments, the solid form comprises the thyroid hormone coating a solid core (such as a solid inert core), which in turn is coated with the coating material. In some embodiments, the solid form consists essentially of the thyroid hormone coated with the coating material.

In some embodiments, the pharmaceutical composition (for example the solid form in the pharmaceutical composition) further comprises a pharmaceutically acceptable excipient, including, but not limited to, a diluent, a filler, a binder, a disintegrant, a lubricant, a preservative, a plasticizer, a coloring agent, or a combination thereof. In some embodiments, the pharmaceutical composition is essentially free of an oxygen scavenger. Exemplary diluents and fillers that can be included in the pharmaceutical composition are modified carbohydrates such as microcrystalline cellulose or inorganic materials such as calcium phosphates. Exemplary binders than can be included in the pharmaceutical composition are pvp, polyvinylpyrrolidone, povidone, methylcellulose, dextrate, and others. Lubricants that can be included in the pharmaceutical composition are, e.g., magnesium stearate, calcium stearate, stearic acid, hydrogenated vegetable oil, mineral oil, glyceryl palmitostearate (Precirol), glyceryl behenate (Compitrol 888):, talc, or fumed silicon dioxide. The pharmaceutical composition can include preservative such as, e.g., cresylic acid, ethyl paraben, and/or benzioc acid. Exemplary plasticizers include, e.g., acetyl tributyl cutratem acetyl triethyl citrate, castor oil, diacylated monoglycerides, dibutyl sebacate, diethyl sebacate, phthalates, etc.

In some embodiments, the thyroid hormone is no more than about any of 50%, 40%, 30%, 20%, 10%, 9%, 8%, 7%, 6%, 5%, 4%, 3%, 2%, or 1% by weight of the total weight of the formulation. In some embodiments, the thyroid hormone is no less than about any of 1%, 2%, 3%, 4%, 5%, 6%, 7%, 8%, 9%, 10%, 20%, 30%, 40%, or 50% of the total weight of the formulation. In some embodiments, the thyroid hormone is present in an amount less than 1% w/w based upon the total weight of the formulation, including for example about 0.01% to about 0.30% w/w, about 0.03% to about 0.25% w/w, about 0.06% to about 0.20% w/w of the total weight of the formulation.

In some embodiments, the thyroid hormone in the solid form is no more than about any of 50%, 40%, 30%, 20%, 10%, 9%, 8%, 7%, 6%, 5%, 4%, 3%, 2%, or 1% by weight of the total weight of the solid form. In some embodiments, the thyroid hormone in the solid form is no less than about any of 1%, 2%, 3%, 4%, 5%, 6%, 7%, 8%, 9%, 10%, 20%, 30%, 40%, or 50% of the total weight of the solid form. In some embodiments, the thyroid hormone is present in an amount less than 1% w/w based upon the total weight of the solid form, including for example about 0.01% to about 0.30% w/w, about 0.03% to about 0.25% w/w, about 0.06% to about 0.20% w/w of the total weight of the solid form.

Also provided herein are solid unit dosage forms comprising a thyroid hormone coated with a coating material. In some embodiments, the unit dosage form comprises about 0.025 mg to about 0.3 mg, such as about 0.025 mg, about 0.03 mg, about 0.035 mg, about 0.04 mg, about 0.045 mg, about 0.05 mg, about 0.055 mg. about 0.06 mg , about 0.065 mg , about 0.07 mg , about 0.075 mg , about 0.08 mg, about 0.085 mg, about 0.09 mg, about 0.1 mg, about 0.125 mg, about 0.13 mg, about 0.135 mg, about 0.14 mg, about 0.145 mg, about 0.15 mg, about 0.155 mg. about 0.16 mg , about 0.165 mg , about 0.17 mg , about 0.175 mg , about 0.18 mg, about 0.185 mg, about 0.19, about 0.2 mg, about 0.225 mg, about 0.23 mg, about 0.235 mg, about 0.24 mg, about 0.245 mg, about 0.25 mg, about 0.255 mg. about 0.26 mg , about 0.265 mg , about 0.27 mg , about 0.275 mg , about 0.28 mg, about 0.285 mg, about 0.29, or about 0.3 mg of thyroid hormone, including any range in between these values. In some embodiments, the unit dosage form is in the form of tablet. In some embodiments, the unit dosage form is in the form of a capsule. In some embodiments, the thyroid hormone in the unit dosage form is no more than about any of 50%, 40%, 30%, 20%, 10%, 9%, 8%, 7%, 6%, 5%, 4%, 3%, 2%, or 1% by weight of the total weight of the unit dosage form. In some embodiments, the thyroid hormone in the solid form is no less than about any of 1%, 2%, 3%, 4%, 5%, 6%, 7%, 8%, 9%, 10%, 20%, 30%, 40%, or 50% of the total weight of the unit dosage form. In some embodiments, the thyroid hormone is present in an amount less than 1% w/w based upon the total weight of the unit dosage form, including for example about 0.01% to about 0.30% w/w, about 0.03% to about 0.25% w/w, about 0.06% to about 0.20% w/w of the total weight of the unit dosage form.

In some embodiments, there are provided articles of manufacture comprising the unit dosage forms described herein. For example, in some embodiments, there is provided a vial (such as a sealed vial or an unsealed vial) comprising a solid form comprising a thyroid hormone coated with a coating material. Other articles of manufacture are also included.

Further provided are batches (such as commercial batches) comprising a solid form comprising a thyroid hormone coated with a coating material. In some embodiments, the batch (such as commercial batch) comprises about 10 μg/ml, about 15 μg/ml, about 20 μg/ml, about 25 μg/ml, about 30 μg/ml, about 35 μg/ml, about 40 μg/ml, about 45 μg/ml, or about 50 μg/ml of thyroid hormone, including any range in between these values. In some embodiments, the thyroid hormone in the batch is no more than about any of 50%, 40%, 30%, 20%, 10%, 9%, 8%, 7%, 6%, 5%, 4%, 3%, 2%, or 1% by weight of the total weight of the batch. In some embodiments, the thyroid hormone in the batch is no less than about any of 1%, 2%, 3%, 4%, 5%, 6%, 7%, 8%, 9%, 10%, 20%, 30%, 40%, or 50% of the total weight of the batch. In some embodiments, the thyroid hormone is present in an amount less than 1% w/w based upon the total weight of the batch, including for example about 0.01% to about 0.30% w/w, about 0.03% to about 0.25% w/w, about 0.06% to about 0.20% w/w of the total weight of the batch. In some embodiments, the batch is essentially free of oxygen scavengers. In some embodiments, the batch is essentially free of an antioxidant.

In some aspects, provided herein is a pharmaceutical composition produced by any of the methods described herein that has substantially no potency loss during storage as compared to the initial potency of the pharmaceutical composition before storage. The initial potency of the armaceutical composition can be evaluated during product manufacturing, before storage or at initial storage. Potency loss can be assayed using any methods known in the art, for example methods described in, but not limited to, Levothyroxine Drug Products: A Review of Clinical and Quality Considerations, Jan. 7, 2013, Medicine and Healthcare products Regulatory Agency (MHRA), UK. As used herein, “substantially no potency loss” indicates the potency loss in the pharmaceutical composition can be less than or equal to about 10% over a desired length of time as compared to the initial potency measurement of the pharmaceutical composition. In some aspects, provided herein is a pharmaceutical composition that has a potency loss of less than or equal to about 10% of the initial potency measurement. In some embodiments, the potency loss is less than or equal to about 9%, about 8%, about 7%, about 6%, about 5%, about 4%, about 3%, about 2%, or about 1%, including any range in between these values, of the initial potency measurement. In some embodiments, the pharmaceutical composition has a potency loss of than or equal to about 10% or any percentage described herein of the initial potency measurement when stored for at least 1 day, 2, days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 30 months, 35 months, 40 months, 45 months, 50 months, 55 months, but no more than 60 months. The pharmaceutical composition provided herein will have substantially no potency loss over a selected time period (e.g., 24 months) when stored under one or more storage conditions that include, but are not limited to, variations in light exposure, variations in temperature, exposure to oxygen, or exposure to moisture. For example, the pharmaceutical composition will have substantially no potency loss when stored over a selected time period (e.g., 24 months) at a temperature of about 4° C., about 8° C., about 10° C., about 15° C., about 20° C., about 25° C., about 30° C., about 35° C., about 40° C., about 45° C., about 50° C., about 55° C., about 60° C., about 65° C., about 70° C., but no more than about 75° C., including any range in between these values.

Thyroid hormone

Thyroid hormones described herein include, but is not limited to: L-3,5,3′,5′-tetraiodothyronine (levothyroxine or LT4); L-3,5,3′-triiodothyronine (liothyronine or LT3); L-3,3′,5′-triiodothyronine (LrT3); L-3,5-diiodothyronine (LT2); or mixtures thereof. As used herein, the term thyroid hormone should be understood to include all pharmaceutically acceptable salts thereof, preferably sodium salts.

In some embodiments, the thyroid hormone is levothyroxine. In some embodiments, the thyroid hormone is levothyroxine sodium. Levothyroxine sodium is the monosodium salt of the levo-rotatory isomer of thyroxine. The chemical name for levothyroxine sodium is sodium (S)-2-amino-3-[4-(4-hydroxy-3,5-diiodphenoxy)-3,5-diiodophenyl]propionate. Levothyroxine (such as levothyroxine sodium) described herein may exist as one or more polymorphic forms, for example one or more crystalline forms, amorphous forms, phases, solid solutions and/or mixtures thereof. In some embodiments, the levothyroxine is in hydrated form, such as the pentahydrate form, quadrahydrate form, dehydrate form, or monohydrate form.

### Coated Material

The methods and formulations described herein uses coating materials. The coating material coats the thyroid hormone and provides a physical barrier that reduces (and in some embodiments prevents) exposure of thyroid hormone to oxygen, light, moisture, and/or heat. In some embodiments, the coating material is substantially water insoluble at neutral pH. In some embodiments, the coating material is impermeable to oxygen. In some embodiments, the coating material is impermeable to light. In some embodiments, the coating material is impermeable to moisture. In some embodiments, the coating material is essentially oxygen-free.

In some embodiments, the coating material dissolves upon digestion. In some embodiments, the coating material dissolves upon exposure to a low pH environment (for example a liquid medium with pH no more than about any of 6.5, 6, 5.5, 5, 4.5, 4, 3.5, 3, 2.5, 2, 1.5, or 1, including any range in between these values). In some embodiments, the coating material is susceptible to enzymatic digestion (such as digestion by enzymes in the digestive system).

In some embodiments, the coating material is essentially gelatin-free. In some embodiments, the coating material has minimal effect on the release profile of the thyroid hormone. For example, in some embodiments, the pharmacokinetic of the thyroid hormone or release profile of the thyroid hormone in the coated formulation is essentially the same as those of a thyroid hormone in an uncoated formulation.

In some embodiments, the coating material has minimal effect on the dissolution profile of the thyroid hormone. For example, in some embodiments, the dissolution profile of the thyroid hormone in the coated formulation is essentially the same as that of a thyroid hormone in an uncoated formulation. In some embodiments, the coating material has minimal effect on the bioavailability of the thyroid hormone. For example, in some embodiments, the bioavailability of the thyroid hormone in the coated formulation is essentially the same as that of a thyroid hormone in an uncoated formulation.

Exemplary coatings that exhibit desired properties described herein are described in, e.g., U.S. 2012/0214917, U.S. 2010/0062062, U.S. 2011/0104277, and U.S. 2011/004828. Commercially available coatings that exhibit desired properties described herein include, e.g., EUDRAGIT® E 12,5, EUDRAGIT® E 100, EUDRAGIT® E PO, Kollicoat® Smartseal 30 D, and OPADRY® fx™.

### Use of the Compositions

The pharmaceutical composition of the present invention can be used in the treatment of hypothyroidism of any etiology, such as primary (thyroidal), secondary (pituitary), or tertiary (hypothalmic) hypothyroidism, except transient hypothyroidism during the recovery phase of subacute thyroiditis. The pharmaceutical composition can also be used to treat subclinical hypothyroidism, in which peripheral thyroid hormone levels are within normal reference laboratory range but serum thyroid-stimulating hormone (TSH) levels are mildly elevated. The pharmaceutical composition can be used for pituitary TSH suppression in the treatment or prevention of various types of euthyroid goiters, thyroid nodules, chronic lymphocytic thyroiditis (Hashimoto's thyroiditis), multinodular goiter and as an adjunct to surgery and radioiodine therapy in the management of thyrotropin-dependent well-differentiated thyroid cancer.

The pharmaceutical composition can be administered in unit dosage form, such as a solid oral dosage in the form of a tablet, capsule, caplet, gelcap, geltab and the like, which can be manufactured according to methods known to those of skill in the art. Exemplary pharmaceutical formulations and/or unit dosage forms of levothyroxine sodium are described in, e.g., U.S. Pat. No. 2,889,363, U.S. Pat. No. 2,889,364, U.S. Pat. No. 3,477,954, U.S. Pat. No. 5,225,204, U.S. Pat. No. 5,324 22, U.S. Pat. No. 5,635,209, U.S. Pat. No. 5,856,359, U.S. Pat. No. 5,955,105, U.S. Pat. No. 6,056,975, and others. The levothyroxine content of the unit dosage form is selected so that oral administration delivers a therapeutically effective dosage of levothyroxine sodium. The amounts required for a specific therapeutic purpose are known to those of skill in the art and can vary widely, based on the desired treatment.

Dosages of the pharmaceutical composition of the present invention can titrated to avoid the consequences of over- or under-treatment. An initial dosage of 12.5 to 50 microcentograms (mcg) orally once daily and can be increased in 12.5 to 25 mcg/day increments every 2 to 4 weeks until the patient with hypothyroidism is clinically euthyroid and/or serum TSH level has normalized. In some embodiments, the dosage thyroid hormone in a unit dosage form in a pharmaceutical composition is between about 12.5 and about 300 mcg, such as about 12.5 mcg, about 25 mcg, about 50 mcg, about 75 mcg, about 88 mcg, about 100 mcg, about 112 mcg, about 125 mcg, about 137 mcg, about 150 mcg, about 175 mcg, about 200 mcg, about 212 mcg, about 225 mcg, about 250 mcg, about 275 mcg, about 288 mcg, or about 300 mcg, including any range in between these values.

### Kits and Unit Dosage Forms

In some aspects, provided herein is a unit dosage form of any of the pharmaceutical compositions disclosed herein. As used herein, “a unit dosage form” can be a tablet, a capsule, or a pill or variations thereof. It is understood that a unit dosage form of the pharmaceutical composition can comprise a stabilized thyroid hormone (e.g., enclosed) with any coating material described herein wherein the pharmaceutical composition is in the shape of a tablet, capsule or a pill. It is further understood that one or more of a unit dosage form described herein can comprise any desired potency or amount of a thyroid hormone. The unit dosage form can be produced by any methods known in the art (see e.g., U.S. Pat. No. 8,361,360; U.S. Pat. No. 8,359,815; and U.S. Pat. No. 7,296,987). For example, a uniform mixture comprising a stabilized thyroid hormone can be compressed into tablets of uniform size and weight using any tableting apparatus known in the art wherein the tablet is subsequently coated with any coating material described herein. In some embodiments, the desired potency or amount of a thyroid hormone produces any desired therapeutic effect described herein. In some embodiments herein, the pharmaceutical composition is in a unit dosage form. In some embodiments, the unit dosage form is a tablet. In some embodiments, the unit dosage form is a capsule. In some embodiments, the unit dosage form is a pill.

Any unit dosage form described herein can be packaged into a container. A container includes, but is not limited to a vial, a bag or a card with cavities for a product. The container may be formed from a variety of materials such as glass or plastic. The container can be sealed or unsealed. The container can also have a removable lid (e.g., a screw top lid) that can be reused. For example, a container with a screw-top lid containing one or more of a unit dosage form described herein can be further sealed with a removable film to prevent exposure of the one or more unit dosage form to oxygen during storage. In some embodiments herein, one or more of a unit dosage form described herein is in a sealed container. In some embodiments herein, one or more of a unit dosage form described herein is in an unsealed container.

In another aspect of the invention, an article of manufacture or kit containing any pharmaceutical composition described herein useful for the treatment and/or prevention of any disorders described above is provided. The article of manufacture or kit can comprise a container containing the pharmaceutical composition described herein and a label or package insert on or associated with the container. The label or package insert indicates that the pharmaceutical composition is used for treating the condition of choice. Moreover, the article of manufacture or kit may comprise (a) a first container with a pharmaceutical composition of the invention; and (b) a second container with a composition contained therein, wherein the composition comprises a further therapeutic agent. The article of manufacture or kit in this embodiment of the invention may further comprise a package insert indicating that the compositions can be used to treat a particular condition.

In some aspects, provided herein is an article of manufacture or kit for coating a thyroid hormone with a coating material described herein useful for the preparation of any pharmaceutical composition described herein. In some embodiments, the article of manufacture or kit can comprise (a) a first container with a coating material described herein; and (b) a label or package insert on or associated with the container. The label or package insert indicates that a composition comprising a thyroid hormone is coated with the coating material to produce a pharmaceutical composition described herein. In some embodiments, the article of manufacture or kit can comprise (a) a first container with a coating material described herein; (b) a second container with a composition comprising thyroid hormone; and (c) a label or package insert on or associated with the container(s). The label or package insert indicates that the thyroid hormone is coated with the coating material to produce a pharmaceutical composition described herein.

We claim:

1. A method of stabilizing a thyroid hormone, preventing a thyroid hormone from being oxidized or degraded, producing a formulation of a thyroid hormone having a uniform potency, or reducing the exposure of a thyroid hormone to oxygen, light, moisture, or high temperature, the method comprising coating said thyroid hormone to provide a solid form comprising said thyroid hormone coated with a coating material.

2. The method of claim 1, wherein the thyroid hormone is levothyroxine or a salt thereof.

3. The method of claim 2, wherein the thyroid hormone is levothyroxine sodium.

4. The method of claim 1, wherein the method comprises an air-suspension encapsulation process.

5. The method of claim 1, wherein the method comprises spraying a solid form comprising thyroid hormone with a composition comprising a pharmaceutically acceptable coating material.

6. The method of claim 1, wherein the method comprises dissolving or dispersing the thyroid hormone in vehicle, and spray coating the dissolved or dispersed thyroid hormone onto an inert solid dosage core.

7. The method of claim 1, wherein the method comprises dissolving or dispersing the thyroid hormone in vehicle and spraying the dissolved or dispersed thyroid hormone into a dessicant system into which a second dispersion comprising a coating mixture is being simultaneously sprayed.

8. The method of claim 1, wherein the method comprises dissolving or dispersing the thyroid hormone in a coating mixture and spraying the dissolved or dispersed thyroid hormone into a dessicant system.

9. The method of claim 1, wherein the coating material is water insoluble at neutral pH.

10. The method of claim 1, wherein the coating material has minimal effect on the dissolution profile of the thyroid hormone.

11. A pharmaceutical composition comprising a solid form of a thyroid hormone, wherein said solid form comprises thyroid hormone coated with a coating material.

12. The pharmaceutical composition of claim 11, wherein the thyroid hormone is levothyroxine.

13. The pharmaceutical composition of claim 12, wherein the thyroid hormone is levothyroxine sodium.

14. The pharmaceutical composition of claim 11, wherein the thyroid hormone is in a hydrated form.

15. The pharmaceutical composition of claim 11, wherein the coating material is water insoluble at neutral pH.

16. The pharmaceutical composition of claim 11, further comprises one or more of a pharmaceutically acceptable excipient selected from the group consisting of: a diluent, a filler, a binder, a disintegrant, a lubricant, a preservative, a plasticizer, and a coloring agent.

17. The pharmaceutical composition of claim 11, wherein the pharmaceutical composition is in a powder form.

18. The pharmaceutical composition of claim 11, wherein the pharmaceutical composition is in the form of a tablet.

19. The pharmaceutical composition of claim 11, wherein the pharmaceutical composition is in the form of a capsule.
